(19) United States
(12) Patent Application Publication
(10) Pub. No.: US 2018/0243127 A1
(43) Pub. Date: Aug. 30, 2018

(54) Modular Cooling Device

(71) Applicant: WeCreate LLC, Culver City, CA (US)

(72) Inventors: Alex Chavarry, Los Angeles, CA (US); Marco Cordova, Santa Fe Springs, CA (US); Colin Greenidge, Thousand Oaks, CA (US); Michael Ochoa, Saugus, CA (US)

(73) Assignee: WeCreate LLC, Culver City, CA (US)

(21) Appl. No.: 15/442,387

(22) Filed: Feb. 24, 2017

(51) Int. Cl. A61F 7/02

(52) U.S. Cl. CPC A61F 7/02; A61F 2007/0011

(57) ABSTRACT

A self-contained modular cooling device vessel having at least one cooling module defined by a pod, a frame, and an integrated connecting means. An insulating substance is contained within at least part of the pod in order to cool a user at temperatures higher than the predetermined temperature of the insulating substance when placing the cooling module proximal to the user's body. The cooling device can include an enclosure that is wearable by the user.

### FIELD OF THE INVENTION

The present invention generally relates to improvements in a self-contained cooling device and the like. The improvements generally relate to providing a modular cooling device that is adaptable, portable, and reusable.

### BACKGROUND

The need exists for a self-contained modular cooling device that is portable, reusable, and adaptable for various uses.

Ice is the most obvious cooling solution whether placed directly in a portable cooler or in an enclosure to apply near the skin for personal cooling. However melted ice leaves an undesirable effect and does not provide reusability. Therefore, a self-contained module is preferable.

Traditionally, many such self-contained personal cooling devices consist of portable fans or misters. However, these devices a traditionally battery-operated, requiring a bulky power source adapted into the device. The problem with such devices requiring a power source is that they are bulky and aesthetically unpleasing to the eye.

An additional problem with personal fans or misters is that the product packaging is limited because nothing cannot interfere with or block the path of the fan or mister, leaving an aesthetically unpleasing product for consumers.

Inventors acknowledge that use of an insulating substance, such as in a refreezable wrap, is an up and coming solution for cooling stored items. However, products incorporating an insulating substance leave significant room for improvement. For example, some refreezable personal cooling devices are formed as a single elongated wrap. To achieve continuous cooling, the user would be required to dedicate large storage space in their freezer to have replacement wraps. The needs exists for an improvement to the reusability.

An additional problem with such cooling devices containing an insulating substance is the structure is often rigid or otherwise unable to be adjusted in size. For example, an elongated wrap may be appropriately sized to be worn around the neck of a user, but inappropriately sized for use around a wrist. Thus, the need exists for a cooling device that is adaptable for various uses. Yet another problem with existing cooling devices is they are comprised of a singular structure which only allows for unidirectional adaptation of the cooling device to the specific use. Therefore a need exists for a cooling device with the ability to multi-directionally adapt for various uses.

Thus, the object of the present invention is to provide improvements to a self-contained modular cooling device that is portable, reusable, and adaptable; and being further capable of easy manufactured, comfortable use, and effective storage.

### SUMMARY OF THE INVENTION

The present invention overcomes the shortcomings associated with old devices and methods, and achieves other advantages not realized by conventional devices and methods.

In view of the foregoing disadvantages in the known types of self-contained modular cooling devices, an objective of this present invention provides a novel cooling device construction for facilitating personal cooling over a period of time for a user susceptible to heating due to the external environment, wherein the same can be adapted for cooling various parts of the body without a need for obtaining a unique device.

Described herein is a self-contained modular cooling device comprised of one or more cooling module, wherein each cooling module comprises a pod, a frame, and a connecting means. An insulating substance is contained within at least part of the pod in order to cool a user whose skin temperature is higher than the predetermined temperature of the insulating substance when worn near the skin. In some embodiments, the cooling device may include an enclosure for shielding a user from at least part of an exterior surface of the pod.

It is an aspect of the present invention to provide a reusable modular cooling device. In the present invention, the pods contain a cooling substance. Once chilled, the pods will be cool for an extended period of time. Upon raising to reaching ambient temperature or above, the pods can been removed from the frame and chilled again for reuse. This modular system allows for space efficient way to provide continuous use, because pre-cooled replacement pods can easily be swapped out for pods that have lost their efficacy due to heat transfer.

The discussed cooling module can be comprised of plastic, metal, glass or any material that is not materially compromised when stored at a low ambient temperature, e.g. in a refrigerator or freezer.

It is an aspect of the present invention to provide a modular cooling device of the types described herein, which accommodates an enclosure so that the device may be worn by the user. A further aspect of the present invention is to provide an aesthetically pleasing cooling device. The cooling device is adaptable to be contained within a variety of enclosures. The enclosures can simply be a compartment in another piece of clothing, i.e. a glove, scarf, sweatband, etc. adapted to receive at least one cooling module. The enclosures also provide a barrier to protect the user's skin from possible ice burn and discomfort, thereby resulting in longer use time. The enclosures can be further adapted to facilitate easy exchange of individual pods. In some embodiments the enclosures may feature a flap that a user can lift to allow direct access to the pods when the temperature of the insulating substance has raised enough to safely apply the pods to the body without the barrier, thereby maximizing the cooling period of the modules.

These and other aspects of the invention are accomplished by a connecting means that allows the cooling module to connect to the enclosures or interconnect to another cooling module allowing to the user to adjust the length of the cooling device and manipulate the shape of the interconnected cooling device in multiple directions. In a preferred embodiment, the connecting means allows the user to interconnect the cooling modules creating an elongate cooling device which can be adapted to a bracelet or a necklace or belt.

The present invention can take a number of different shapes with accompanying functional and/or aesthetic benefits. For example, a plurality of smaller interconnected cooling modules may be preferred to facilitate forming a bracelet to be worn around the wrist. Additionally, the pods can be shaped to achieve various aesthetic or marketing benefits. The component parts of the cooling module can be adapted, configured, or customized to meet particular functional or aesthetic needs of particular users. One may also contemplate the use of this cooling pod as part of a cooling storage device, such as a portable cooler.

Other independent features and advantages of the invention will become apparent from following detailed description, taken in conjunction with the accompanying drawings which illustrate, by way of example, the principles of the invention. Various changes and modifications within the spirit of the invention will become apparent to those skilled in the art.

In this respect, before explaining at least one embodiment of the invention in detail, it is to be understood that the invention is not limited in its application to the details of construction set forth in the following description or illustrated in the drawings. The invention is capable of other embodiments and of being practiced and carried out in various ways or with various materials. Also, it is to be understood that the phraseology and terminology employed herein are for the purpose of description and should not be regarded as limiting.

### BRIEF DESCRIPTION OF THE DRAWINGS

FIG. 1A is a diagrammatic top perspective view of an exemplary embodiment of the present invention.

FIG. 1B is a diagrammatic bottom perspective view of the embodiment of FIG. 1A.

FIG. 2 is a diagrammatic bottom perspective view of an exemplary cooling pod of the present invention.

FIG. 3A is a diagrammatic perspective view of an exemplary frame and connecting means of the present invention.

FIG. 3B is a diagrammatic front view of the embodiment of FIG. 3A.

FIG. 3C is a diagrammatic rear view of the embodiment of FIG. 3A.

FIG. 3D is a diagrammatic left side view of the embodiment of FIG. 3A.

FIG. 3E is a diagrammatic right side view of the embodiment of FIG. 3A.

FIG. 3F is a diagrammatic front sectional view of the embodiment of FIG. 3A.

FIG. 3G is a diagrammatic top view of the embodiment of FIG. 3A.

FIG. 3H is a diagrammatic bottom view of the embodiment of FIG. 3A.

FIG. 4A is a diagrammatic perspective view of an exemplary embodiment of the present invention featuring an alternative configuration.

FIG. 4B is a diagrammatic bottom perspective view of the embodiment of FIG. 4A.

FIG. 4C is a diagrammatic side cross-sectional view of the embodiment of FIG. 4A depicting an exemplary connecting means.

FIG. 4D is a diagrammatic front view of the embodiment of FIG. 4A depicting an exemplary use.

FIG. 5A is a diagrammatic side view of an alternate exemplary embodiment of the present invention depicting an alternative connecting means.

FIG. 5B is a diagrammatic side cross-sectional view of the embodiment of FIG. 5A.

FIG. 6A is a diagrammatic perspective view of a further alternate exemplary embodiment of the present invention depicting an alternative frame configuration.

FIG. 6B is a diagrammatic side cross-sectional view of the embodiment of FIG. 6A.

FIG. 7A is a diagrammatic perspective view of a further alternate exemplary embodiment of the present invention depicting an alternative configuration.

FIG. 7B is a diagrammatic bottom perspective view of the embodiment of FIG. 7A.

FIG. 7C is a diagrammatic perspective view of the embodiment of FIG. 7A depicting the frame.

FIG. 8A is a diagrammatic perspective view of a still further alternate exemplary embodiment of the present invention depicting an alternative configuration.

FIG. 8B is a diagrammatic perspective view of the embodiment of FIG. 8A depicting the frame.

FIG. 9A is a diagrammatic perspective view of a still further alternate exemplary embodiment of the present invention depicting an alternative configuration.

FIG. 9B is a diagrammatic perspective view of the embodiment of FIG. 8A depicting the frame.

FIG. 10A is a diagrammatic top perspective view of an exemplary embodiment of the present invention depicting an exemplary enclosure.

FIG. 10B is a diagrammatic top perspective view of the embodiment of FIG. 10A depicting an alternate configuration.

### DETAILED DESCRIPTION

The following descriptions are exemplary embodiments only, and are not intended to limit the scope, applicability or configuration of the invention in any way. Various changes to the described embodiments may be made in the function and arrangement of the elements described without departing from the scope of the invention as set forth in the appended claims.

The present invention is directed towards a self-contained modular cooling system and a method of personal cooling utilizing an adaptable modular cooling device. With reference to the annexed drawings, the preferred embodiments of the present invention are herein described for indicative purposes and by no means as a limitation.

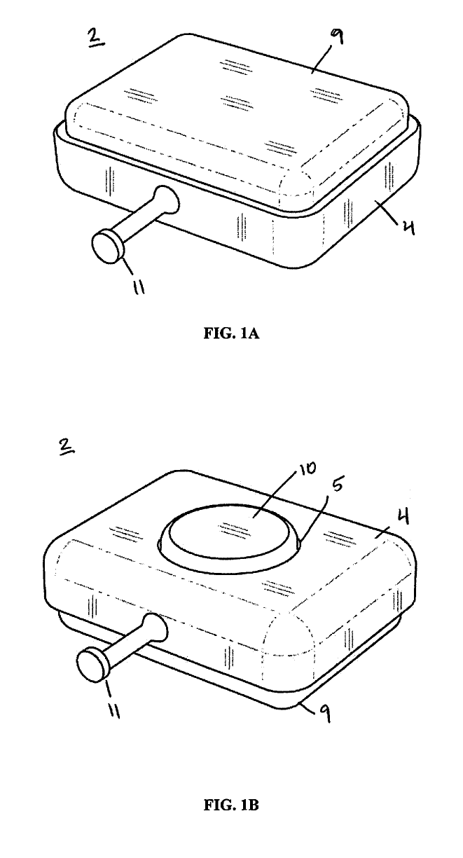

FIGS. 1A and 1B are a top and bottom perspective view, respectively, of an embodiment according to the present invention. The cooling device 1 comprises one or more cooling Modules 2 which comprise a frame 4 having a connecting means 11 and a pod 9.

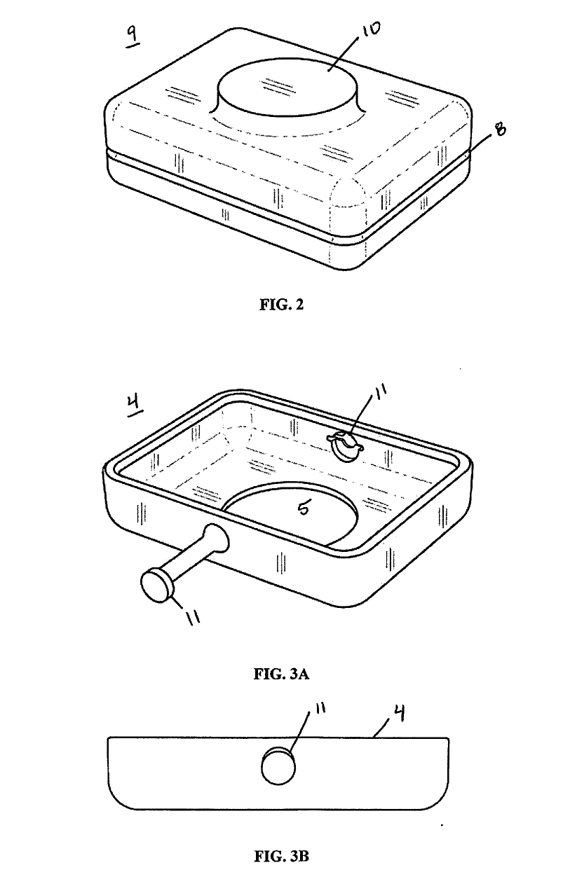

FIG. 2 is a perspective view of the embodiment of FIG. 1A depicting the pod 9, wherein the pod 9 contains an insulating substance 3. A pod 9 substantially filled with the insulating substance 3 is optimal for maximal cooling; however a pod 9 at least partially filled with insulating substance 3 can provide adequate cooling benefits.

The composition of the insulating substance 3 is not a claimed feature of the present invention. The insulating substance 3 in the present invention can be any non-toxic non-flammable substance that is capable of attaining a lowered temperature. Differing compositions of the insulating substance 3 will have varying properties. Common insulating substances 3 are water, hydroxyethyl cellulose, polymer gel, silica gel, etc. Other exemplary insulating substances 3 that are known to those in the art include a mixture of rubbing alcohol or a mixture of sodium chloride, water, and glycol. Some insulating substances will change to a solid phase when frozen, others will remain malleable.

A preferred insulating substance 3 would have a lower freezing point than water, providing for a phase-change of the insulating substance 3 for enhanced absorption of heat over an extended period thereby maintaining a low temperature and a slower rate at which the insulating substance 3 temperature raises to ambient temperature. Freezing points between 32 degrees and 10 degrees Fahrenheit are preferred.

One skilled in the art may enhance features of the insulating substance 3 with sodium chloride, minerals, water, or other additives. For aesthetic purposes, visual appearance of the insulating substance 3 can be enhanced, for example with dye or other decorations.

The preferred method of use for the present invention involves placing the cooling device 1 on or near the user's body to cool a local area of their body. Such use is achieved by placing a pre-cooled pod 9 that has preferably reached a minimum cooling temperature in a frame 4 and either interconnecting a plurality of frames 4 or connecting one or more frames 4 to an enclosure 12 so the user can wear the cooling device 1 near the skin. The temperature of the insulating substance 3 rises slowly to ambient temperature influenced both by the heat the item surrenders to the insulating substance 3 and the heat of the ambient. Therefore, the user will be cooled as their body heat is absorbed by the insulating substance 3 due to the close proximity of the cooling device 2 to the skin. The proximity of the cooling device 1 to the skin may vary but shall be close enough to allow the insulating substance 3 to absorb at least some of the user's body heat. In a preferred embodiment, the proximity would be close enough such that the insulating substance 3 absorbs more heat from the user's body than from the environment.

An alternative embodiment of the present invention may feature a pod 9 comprising an access point 10 to provide the user access to fill the pod 9. For example, if the insulating substance 3 is water. One skilled in the art can appreciate how such an embodiment could provide for ease of shipping.

The pod 9 may be constructed of various and multiple materials. The pod 9 can be made from any non-porous material that is resistant to deformation and are not compromised when stored in frigid and wet conditions for extended periods of time, including plastic, metal, or glass. The pod 9 can be enhanced by using any material less permeable to heat transfer to minimize heat exchange from the surroundings to the insulating substance, thereby extending the cooling period of the pod 9. Additionally, flexible materials may be optimal for any composition of insulating substance 3 that expands when its temperature is lowered.

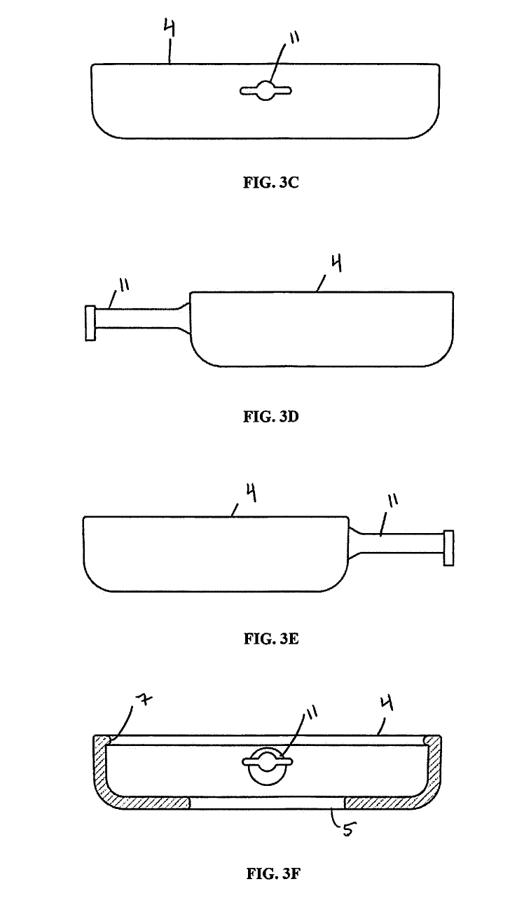

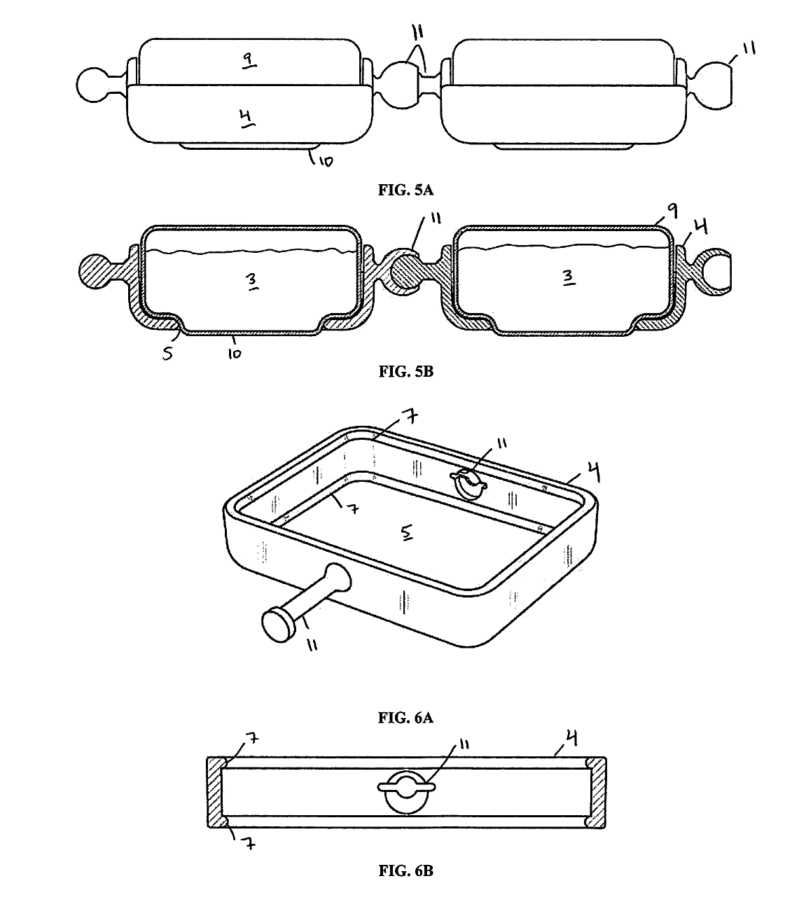

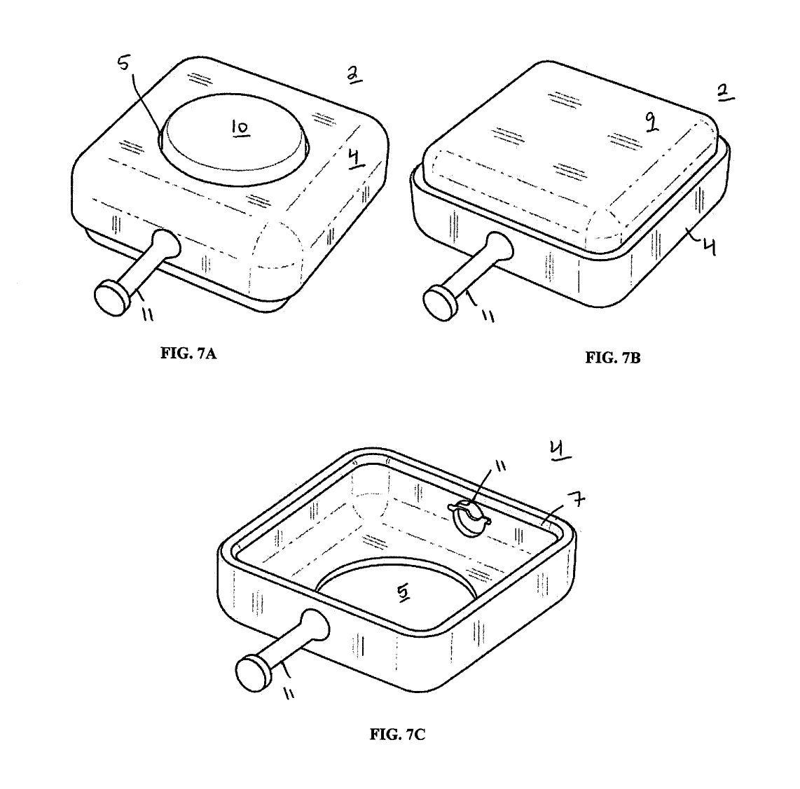

FIGS. 3A-3H are views of the embodiment of FIG. 1A depicting the frame 4 and the connecting means. In the present invention, the frame 4 may be a case or any other construction designated and adapted to removably receive and hold the pod 9. In some embodiments, the frame 4 can be configuration to expose more or less of the pod 9 to the user. In embodiments where the frame 4 substantially cover the pod 9 could protect the pod 9 or provide enhanced grip to the user. FIG. 1A depicts one embodiment of the present invention having one surface of the pod 9 exposed and an aperture 5 in the opposite surface. In such an embodiment, one face of the cooled pod 9 will be exposed to the heat transfer from the user's skin. Once the insulating substance 3 adjacent to that face lowers to an ineffective temperature, the user can flip the pod 9 over to utilize the cooling characteristics of the insulating substance 3 adjacent to the other side of the pod 9. Additionally, the aperture 5 provides the user with a method for easy removal of the pod 9. FIG. 7A depicts another exemplary embodiment wherein the frame 4 is configured as a rigid band over the perimeter of the pod 9, thereby exposing the user to the cooling attributes of a most of the surface area of the pod 9. One skilled in the art may enhance the features of the frame 4 to provide for ease of pod 9 removal. For example, FIGS. 8A and 8B depict an exemplary embodiment wherein the frame 4 comprises a Slit 6 to provide access to the pod 9 for leveraging its removal. Other embodiments of the frame 4 may provide for maintaining a preferred spatial separation between the frame 4 and the pod 9 received therein.

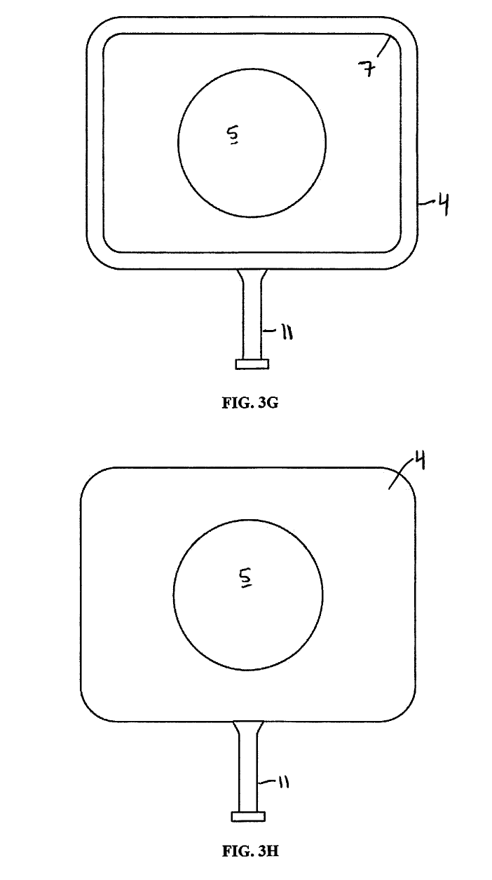

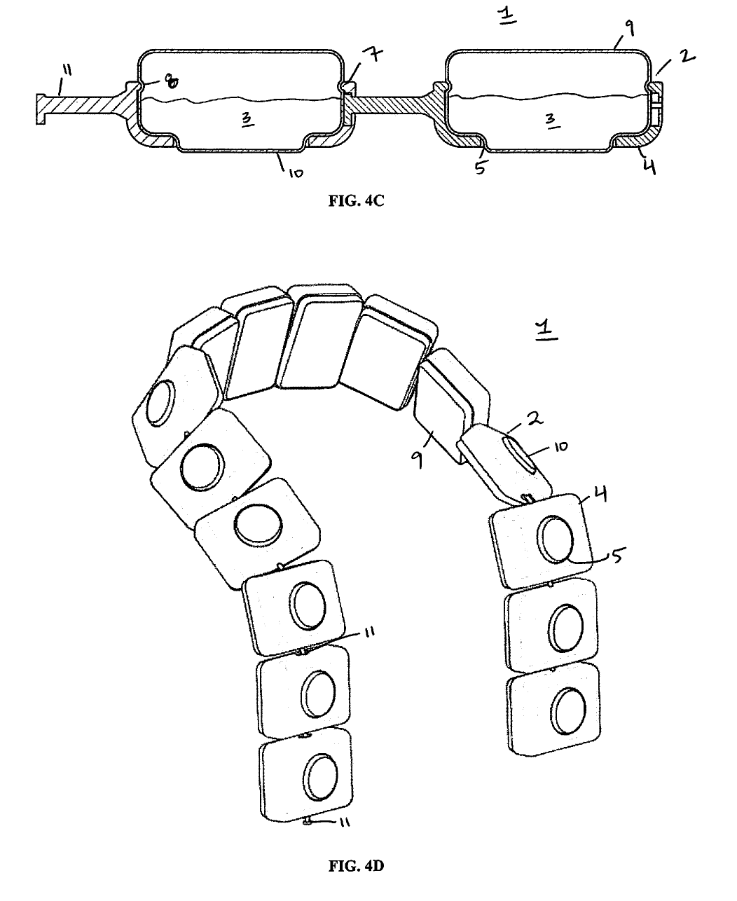

In the present invention, the pod 9 is removably received by the frame 4, meaning the cooling device 2 components shall be configured to allow the frame 4 to hold the pod 9 in place without user assistance, yet allow the user to remove the pod 9 from the frame 4 when necessary to place the pod 9 in a fridge, freezer, cooler, etc. In a preferred embodiment, the pod 9 and frame 4 shall be removably engaged such that the pod 9 can be inserted and removed without excessive force. FIG. 2 depicts a preferred embodiment of the invention featuring a configuration in which the pod 9 has a groove 8 along its perimeter. In direct relation, FIG. 3A depicts an embodiment of the invention featuring a configuration in which the frame 4 features a lip 7 located along an inside edge of the frame 4. In such an embodiment, the groove 8 of the pod 9 is intended to receive the lip 7 of the frame 4 creating a removable engagement between these components, such as a snap-fit connection, thereby holding the pod 9 in place within the frame 4 as depicted in FIG. 4C. Alternate embodiments of the invention may include a configuration featuring an alternative lip 7 placement. FIGS. 6A and 6B depicts an alternate preferred embodiment wherein the frame 4 features two lips 7 protruding from the upper and lower inner edges of the frame 4. In such a configuration, the corresponding pod 9 may feature two corresponding grooves 8 to receive each lip. Alternatively, the frame 4 may receive the pod 9 such that the entire side surfaces are covered by the frame 4 and the top and bottom faces of the pod 9 are exposed, but flush with the lips 7 of the frame 4.

The frame 4 may also be constructed of various and multiple materials allowing for removable receipt of the pod 9. In some embodiments, the frame 4 can be constructed of a rigid elastomer, silicone, or metal allowing the pod 9 to snap fit into the frame 4. In another embodiment, the frame 4 may be comprised of a flexible elastomer wherein the inherent retractability of the frame 4 provides the mechanism to hold the pod 9 in place. In some preferred embodiments, the frame 4 can also be enhanced by using any material less permeable to heat transfer to minimize heat exchange from the surroundings to the cooled pod 9, thereby extending the cooling period of the pod 9. In other embodiments, the frame 4 can comprise of any of a number of versatile materials selected to be capable of shielding a user from a cold surface, including thermoplastic, elastomers, foam resin, thermoset rubber, silicone, treated leather, polyurethane, neoprene, or synthetic polyester and nylon materials.

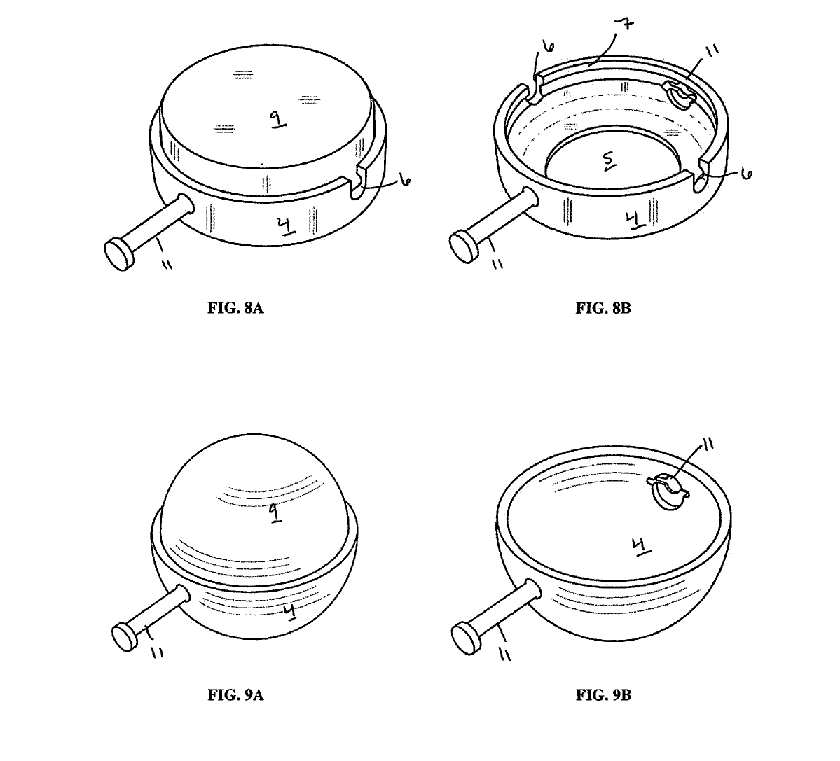

The components of the cooling device 2 may be configured in various shapes with due to manufacturing and/or design preferences. FIGS. 1-3 depict a pod 9 substantially shaped as a rectangular prism or a cuboid wherein each face of the pod 9 is substantially rectangular. FIGS. 7-9 are sectional views of exemplary embodiments of the present invention featuring alternative pod 9 configurations. FIGS. 7A-7C depict a pod 9 with an alternative cuboid shape, wherein one face is substantially square, and its corresponding frame 4. FIGS. 8A and 8B depict a pod 9 shaped as a cylinder and a corresponding frame 4, respectively. FIGS. 9A and 9B depict a pod 9 shaped as a cube and a corresponding frame 4, respectively. Other embodiments may comprise of any other shape.

The present invention features a connecting means 11 integrated into the frame 4 to allow a user to interconnect the cooling Modules 2 to provide adaptability and adjustability cooling device. The cooling Modules 2 may be interconnected in various arrangements to serve its intended purpose. FIGS. 4A and 4B depict an exemplary embodiment of the present invention featuring a plurality of cooling Modules 2 interconnected to form a linear configuration that may be adjusted in multiple directions. FIG. 4D depicts an exemplary embodiment that may be used to drape the cooling device 1 around the neck of a user. This arrangement of the connecting means 11 on opposing sides of the frame 4 may be preferable when connecting the cooling Modules 2 to form a linear chain or a loop, such as a scarf or necklace. Alternatively, arranging the connecting means 11 on adjacent sides of the frame 4 may be utilized to connect the cooling device 2 into different shapes.

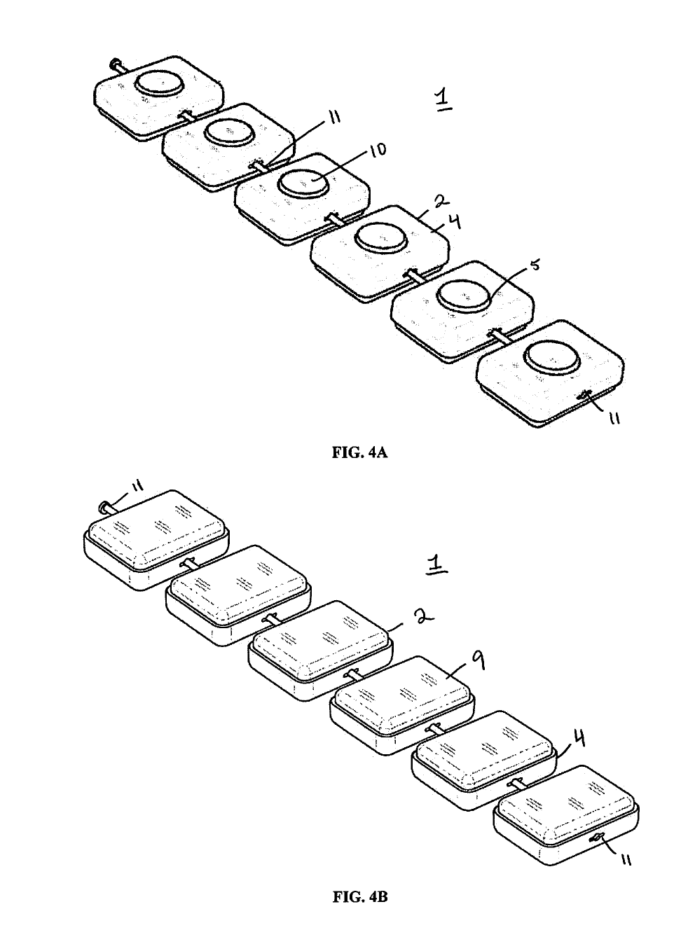

The cooling device 1 may be comprised of various connecting means 11 integrated into the frame 4 to interconnect the cooling modules. In a preferred embodiment of the present invention, the connecting means 11 is comprised of a quick connect coupling means for ease of use providing an adjustable cooling device. FIGS. 1A-4 depict a preferred embodiment wherein the connecting means 11 comprises a male connector protruding from the one side of the frame 4 and a female socket integrated into the opposite side of the frame 4. Other exemplary embodiments may include a ball and socket connecting means 11 comprising a socket member and ball member protruding from opposite sides of the frame 4 as depicted in FIGS. 5A and 5B. Yet another exemplary embodiment may include hook and eye connectors or a rod button connection. The use of various connectors may be apparent to one skilled in the art. In a preferred embodiment the connecting means 11 allows partial flexibility for the frames 4 to at least partially maneuver about one another.

The present invention may also feature an enclosure 12 adapted to receive at least one cooling module 2. In a preferred embodiment the enclosure 12 may be comprised of an article of clothing or a compartment therein with at least one opening 14 to receive a plurality of interconnected cooling modules. Such an enclosure 12 may be further adapted with a the same connecting means 11 in order to secure the cooling module(s) 2 therein to prevent shifting or other unwanted movement within the enclosure. Based on user preference, the enclosure 12 may also be configured with a fastening means to fully or partially close off the opening, thereby further enclosing the cooling module(s) 2. Alternatively, the enclosure 12 may be configured with at least one opening 14 intended to remain open to allow the user free access to the cooling module(s) 2 or to allow the user to slide the cooling modules 2 in and out with ease.

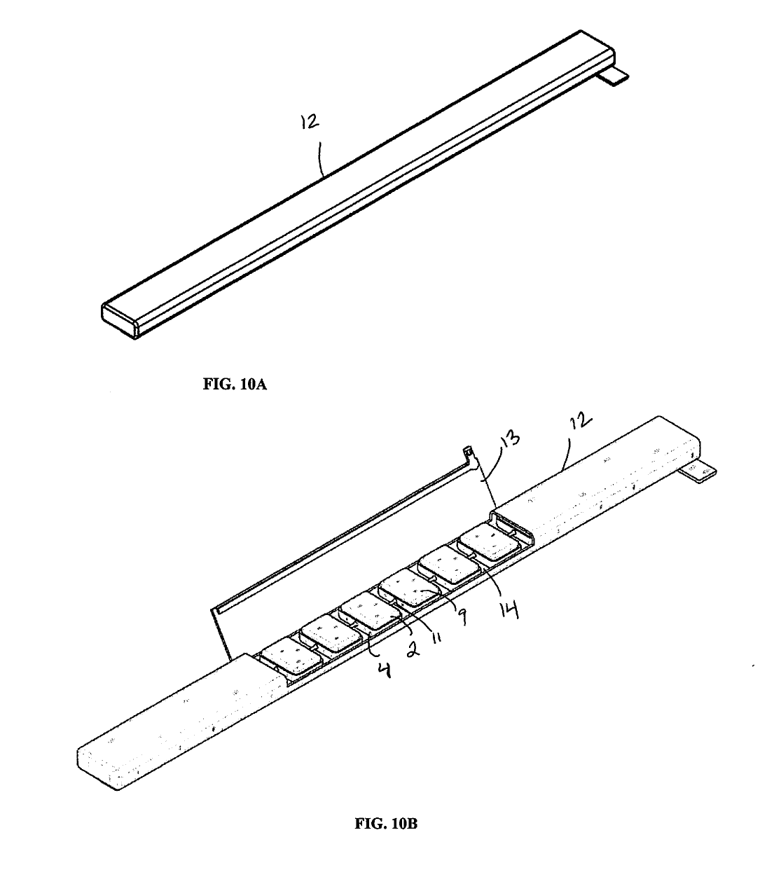

FIGS. 10A and 10B depict exemplary embodiments wherein a sleeve is adapted to receive a linear chain of interconnected cooling modules 2. Such an embodiment may be worn by the user as a scarf or neck wrap. FIG. 10A depicts an exemplary elongated enclosure 12 that opens and closes along its length to provide easy removal of the entire chain and to fully enclose the chain when closed. FIG. 10B depicts an alternative embodiment wherein the enclosure 12 features an opening 14 comprising an access panel 13 over part of its length to provide easy access to exchange individual pods 9 upon losing its cooling effect. In a preferred embodiment, the access panel 13 is slightly wider than the opening 14 in the sleeve, such that when the access panel 13 is closed, the overlap provides extra support and an aesthetically appealing finish. In such an embodiment, when the cooling properties of the pod 9 have lowered, but the insulating substance 3 is still below ambient, a user can simply lift the access panel 13 and allow the pod 9 to be place directly in close proximity with the user's body, rather than accessing the opening 14 configured to access the entire chain of cooling modules. In alternative embodiments, the access panel 13 can be located based on which cooling Modules 2 may encounter the most heat transfer, e.g. those placed in closest proximity to parts of the body that are prone to exert more heat, and would therefore need to be replaced more frequently than other interconnected cooling modules. For example, in the embodiment depicted in FIG. 10B is intended to be draped around a user's neck, therefore the cooling Modules 2 in the middle will likely be placed on the back of the user's neck and would lose their cooling efficacy more rapidly than the other peripheral cooling modules 2.

In such an embodiment, various fastening means may be used to at least partially close the opening 14 of the enclosure, such as a zipper, Velcro, buttons, or a hook and eye fastener. In a preferred embodiment the fastening means should be able to withstand repeated use, easy to use, temporary and capable of securely staying fastened, but still able to be unfastened without having to exert sufficient force.

In an alternative embodiment, the enclosure 12 may be comprised of a compartment within a glove adapted to receive a single cooling module 2. Other embodiments may include a compartment in a shirt, a belt, a bracelet, etc. The enclosure 12 can comprise of any of a number of versatile materials selected to serve its intended purpose. In a preferred embodiment, the enclosure 12 is comprised of a cloth material capable of permeating the cooling attributes of the cooling device. In some embodiments, the enclosure 12 may be partially comprised of a material capable of shielding the cold surface of the pod 9 from the environment.

Combinations of the above listed types of arrangement could be used within a cooling device 1 depending on the specific needs and/or requirements for the vessel. The exterior surface of the pod 9 and/or frame 4 can be printed or embossed with advertising, marketing, branding logos, etc.

It is understood that the present invention is not limited to the embodiments described within the scope of the following claims.

What is claimed is:

1. A modular cooling device comprising:
at least one cooling module comprising:
a pod containing an insulating substance;
a frame adapted to removably receive at least part of said pod; and
a connecting means integrated in to said frame.

2. The modular cooling device of claim 1 wherein said connecting means comprises a male member and a female socket.

3. The modular cooling device of claim 1 wherein said connecting means comprises a ball and socket connection.

4. The modular cooling device of claim 1 wherein said connecting means comprises a hook and eye connection.

5. The modular cooling device of claim 1 further comprising an enclosure comprising at least one opening adapted to removably receive said at least one cooling module;
wherein said enclosure is adapted to be worn on the body of a user.

6. The modular cooling device of claim 5 wherein said enclosure comprises a connector adapted to engage said connecting means.

7. The modular cooling device of claim 5 wherein said enclosure at least partially comprises a cloth material.

8. The modular cooling device of claim 1 wherein said frame comprises a band configuration adapted to removably receive at least part of a periphery of said pod.

9. The modular cooling device of claim 1 wherein said frame comprises at least one inwardly protruding lip adapted to engage with at least part of an exterior surface of said pod.

10. A method of cooling a user according to claim 1, said method comprising:
lowering said insulating substance temperature below ambient temperature; and
placing said cooling device in close proximity to the body of the user.

11. The modular cooling device of claim 1 wherein said pod is substantially cuboidal in shape.

12. The modular cooling device of claim 1 wherein said pod has a substantially circular cross section.

13. A modular cooling device comprising:
a plurality of cooling modules, each said cooling module comprising:
a pod containing an insulating substance;
a frame adapted to removably receive at least part of said pod; and
a connecting means integrated in to each said cooling module;
wherein said at least one of said plurality of cooling modules is removably connected to an adjacent said cooling module.

14. The modular cooling device of claim 13 wherein said connecting means comprises a first and second connector protruding from said frame in substantially perpendicularly directions from each another.

15. The modular cooling device of claim 13 wherein said connecting means comprises a first and second connector protruding from said frame in substantially opposing directions from each another.

16. The modular cooling device of claim 13 further comprising an enclosure adapted to removably receive said plurality of cooling modules, wherein said enclosure is adapted to be worn on the body of a user.

17. The modular cooling device of claim 16 wherein said enclosure comprises an elongate configuration wherein said enclosure is worn around the neck of a user.

18. The modular cooling device of claim 13 wherein at least one of said frame comprises at least one inwardly protruding lip adapted to engage with at least part of an exterior surface of at least one of said pod.

19. A method of cooling a user according to claim 13, said method comprising:
lowering said insulating substance temperature below ambient temperature; and
placing said cooling device in close proximity to the body of the user.

20. A modular cooling device comprising:
a plurality of cooling modules, each said cooling module comprising:
a pod containing an insulating substance;
a frame adapted to removably receive at least part of said pod; and
a connecting means integrated in to each said cooling module;
an elongate enclosure, configured to at least partial cover said plurality of cooling modules, comprising:
at least one opening adapted to removably receive said plurality of cooling modules;
an interior surface; and
a connector integrated to said interior surface, wherein said connector is adapted to engage said connecting means;
wherein at least one of said plurality of cooling modules is removably connected to said elongate enclosure;
wherein each remaining said plurality of cooling modules is removable connected to an adjacent said cooling module.

21. The modular cooling device of claim 20 wherein said frame comprises a planar rim protruding inwardly and said pod comprises a planar groove corresponding to said planar rim to facilitate a removable engagement between said frame and said pod.

22. The modular cooling device of claim 20 wherein said enclosure further comprises at a fastening means to at least partially close said at least one opening.

23. A method of cooling a user according to claim 20, said method comprising:
lowering said insulating substance temperature below ambient temperature; and
placing said cooling device in close proximity to the body of the user.
